(19) United States
(12) Patent Application Publication
(10) Pub. No.: US 2003/0099613 A1
(43) Pub. Date: May 29, 2003

(54) Viral replicons and viruses dependent on inducing agents

(76) Inventors: Benjamin Berkhout, Naarden (NL); Koenraad Dirk Verhoef, Witney Oxon (GB)

Correspondence Address: TRASK BRITT, P.O. BOX 2550, SALT LAKE CITY, UT, 84110, US

(21) Appl. No.: 10/097,542

(22) Filed: Mar. 8, 2002

Related U.S. Application Data
(63) Continuation of application No. PCT/NL00/00637, filed on Sep. 8, 2000.

(30) Foreign Application Priority Data
Sep. 10, 1999 (EP) EP 99202971.0

(51) Int. Cl. A61K 48/00; C12N 7/00; C12N 15/86

(52) U.S. Cl. 424/93.2; 435/456; 435/235.1

(57) ABSTRACT

An inducible viral replicon comprising at least one inducible repressor and/or activator, and all viral sequences which are essential for replication under direct or indirect control of said inducible repressor and/or activator. The inducible repressor and/or activator may comprise a Tet operon. A replicon of the invention may be used to produce a virus, for instance an attenuated HIV virus, which can be induced to replicate by the presence of doxycycline or a functional analog thereof. The replicon and/or the produced virus may be used to prepare a vaccine, for instance for the prophylaxis of AIDS. A replicon of the invention can be modified, preferably improved, by culturing the replicon in permissive cells for an extended period of time.

### RELATED APPLICATIONS

This application is a continuation of co-pending International Application No. PCT/NL00/00637, filed Sep. 8, 2000, designating the United States of America and corresponding to International Publication No. WO 01/20013 A2, the contents of the entirety of which are incorporated by this reference, which itself claims priority from EP 99202971.0, filed on Sep. 10, 1999.

### TECHNICAL FIELD

The present invention relates to the field of molecular biology of pathogens, in particular viruses, and more in particular to human immunodeficiency virus. It relates to methods for producing replicons and/or viruses dependent on inducing agents, the replicons and/or viruses, as well as uses of such replicons and/or viruses in the production of vaccines, and in particular to live-attenuated vaccines.

### BACKGROUND

Live-attenuated virus vaccines (such as vaccinia, polio and measles) have been enormously successful and have made a dramatic and historic impact on public health. However, for the human immunodeficiency virus type 1 (HIV-1), safety concerns remain about either the reversion of attenuated vaccine strains to virulent phenotypes or the induction of fulminant infection in immunocompromised individuals.

Testifying to the genetic instability of such strains is the recent demonstration that the HIV-1 delta3 vaccine candidate, which contains 3 deletions in non-essential parts of the genome, is able to regain full replication capacity within four months of replication in tissue culture (Berkhout et al., 1999). In addition, it has been recently reported that replication of deletion variants of the simian immunodeficiency virus (SIV) increased after several years in some infected monkeys, concomitant with the onset of AIDS (Baba et al., 1999). Furthermore, although there is some evidence that attenuated HIV-1 variants lacking the nef gene result in a benign course of infection in humans (Deacon et al., 1995), a decline in CD4+ T-cell numbers has been reported recently for some of these individuals, which is an early sign that these persons could develop AIDS (Dyer et al., 1999; Greenough et al., 1999). These results have forced the development of subunit or inactivated virus vaccines. However, these vaccines have not elicited the potent broad-based immune responses or long-term memory necessary to confer life-long protection in immunized individuals (reviewed in Paul, 1995). Accordingly, live-attenuated HIV vaccine approaches are still being considered.

Replicating virus vaccines demonstrated superior performance in AIDS vaccine trials. It has been repeatedly demonstrated that macaques or chimpanzees persistently infected with genetically attenuated, non-pathogenic isolates of SIV or HIV-1 respectively, strongly resist a subsequent challenge with pathogenic virus (Shibata et al., 1997; Wyand et al., 1996; van Rompay et al., 1995; Almond et al., 1995; Daniel et al., 1992; Lohman et al., 1994; Stahl-Hennig et al., 1996; Johnson et al., 1999). However, to satisfy safety concerns, the ideal vaccine strain should replicate only to the extent that is needed for immunogenicity.

### DISCLOSURE OF THE INVENTION

Towards the construction of the next generation of safe, genetically stable, HIV-1 variants as a live-attenuated AIDS vaccine, the present invention discloses the construction of a HIV-1 variant of which the replication depends on the addition of an inducing agent such as the non-toxic, selective effector doxycycline (dox). Thus, the invention provides an inducible viral replicon, comprising at least one inducible repressor and/or activator, and all viral sequences which are essential for replication under direct or indirect control of the inducible repressor and/or activator.

In one embodiment, at least part of the viral sequences in the inducible replicon is RNA.

The invention is exemplified by embodiments relating to Human Immunodeficiency Virus (HIV). However, the invention will be applicable to other pathogens, in particular viral pathogens, of which it is important that they replicate in order to obtain an efficacious immune response, but for which it is also important that the replication does not go beyond the level required for an immune response.

A replicon is defined as a nucleic acid molecule capable of replication in a suitable environment, such as a permissive cell, because it has all the necessary elements for replication in such an environment. We call it a “replicon,” because it will not always be directly derived from the nucleotide sequences of the original pathogen, for instance in the case of single stranded DNA viruses, RNA viruses, etc. Typically, in order to manipulate nucleic acids, double stranded forms of the nucleic acid are necessary, such as double stranded DNA. Therefore, preferred replicons will be double stranded DNA nucleic acids in at least one stage of their life cycle.

A replicon is also intended to reflect that the actual pathogen, or its attenuated live vaccine relative, usually comprises more than just a nucleic acid. The nucleic acid is typically packaged into a (viral) particle. Therefore, the replicon also encodes a functional packaging signal, allowing for the nucleic acid in its wild-type-like form (RNA in the case of a retrovirus, etc.) to be packed into a viral particle. In order for the replicon to be able to replicate in a host, it is desirable that the replicon also carries the structural genes for the proteins of the envelope and/or capsid, be it in wild-type format or in a somewhat different format (reduced or enhanced target binding, etc.).

In order to be able to regulate the amount of replication necessary for eliciting a good immune response without any replication beyond that level, at least one gene essential for the replication is placed under the control of an inducible repressor/activator according to the present invention. In order to prevent leakage, it is desirable to have a combination of essential genes under such control, and it is even more desirable to have at least two different repressor/activator combinations in control of at least one, but preferably more than one, gene essential for replication. In most (viral) pathogens, a number of genes is essential for replication, but most of them also have a sort of “master switch”, such as an early gene that transactivates other genes. A first candidate to put under direct control of a repressor/activator is such a master switch, which indirectly provides control over the other essential genes for replication. Still, it is preferred to put at least one other essential gene under control of an inducible repressor/activator. However, a master switch is not required for ‘simple’ viral genomes such as HIV-1 that are under control of a single transcription unit.

As stated previously herein, the replicon is preferably a viral replicon which is derived from a human immunodeficiency virus. Typically, such a replicon would be an infectious double stranded DNA clone of an HIV strain. Preferably, the HIV strain is an attenuated strain or is made into an attenuated strain by introducing mutations, such as functional deletions as those described herein. Any repressor/activator elements that are inducible are applicable in the present invention. Typically, when they are used as a single element, the repressor/activator elements should not have leakage (meaning low base levels of gene expression) in the repressed or unactivated state. In the case of double or more inducible controls, the leakage becomes less important, although essentially no leakage is still highly preferred.

A good system for inducible control is the combination of the Tet-operon and doxycycline as the inducing agent. Thus, the invention also provides a viral replicon wherein the inducible repressor and/or activator comprises a Tet operon or a functional equivalent thereof. This operon and its necessary elements are known to those of ordinary skill in the art and are further described hereinafter. A functional equivalent thereof is an element that is capable of repression and/or activation in essentially the same manner as the Tet operon. Typically, this would be highly homologous variations of the Tet operon. As a safety valve, it would be advantageous to provide the replicon with a suicide gene that can be activated when unwanted effects occur, such as replication beyond what is necessary for an immune response or rescue by wild type virus, etc. As example of a suicide gene is HSV-tk, which may be induced by adding gancyclovir or a functional equivalent thereof. Upon induction, the gene will kill the infected cell, and thereby inhibit further replication and infection of other cells. Thus, in yet another embodiment, the invention provides a replicon according to the present invention which further comprises a suicide gene.

As stated previously herein, the replicon is preferably under control of at least a Tet operon which allows for replication in the presence of doxycycline. Thus, the invention also provides a replicon according to the invention which can be induced to replicate by the presence of doxycycline or a functional analog thereof.

In the present context, a functional analog of doxycycline is a molecule capable of removing repression or initiating activation of the genes under control of the activator and/or repressor present in the replicon.

In order to attenuate the HIV replicon and/or the resulting virus, it is preferred that the replicon is provided with a functional deletion of the TAR-element. Thus, in yet another preferred embodiment, the invention provides a replicon according to the present invention which further comprises an inactivated TAR element.

In order to attenuate the HIV replicon according to the invention, it is preferred to functionally delete the Tat element. Thus, the invention also provides a replicon according to the present invention which further comprises an inactivated Tat element. Preferably, both elements mentioned above are functionally deleted. “Functional deletion” means that at least their function in the replication of the replicon is at least partially inhibited. Essential genes for replication typically should not be completely dysfunctional. Proteins necessary for removing repression or initiating activation elements which are present upstream of the essential genes to be put under control should be encoded by the replicon and inserted in a non-essential gene. Thus, the invention also provides a replicon according the present invention wherein at least one functional part, such as an rtTA gene, of the inducible repressor and/or activator is inserted into the nef gene. The functional part in this case refers to any proteinaceous substance capable of activating or derepressing the element in control of the essential gene. Preferably, space is created for the sequence encoding the proteinaceous substance. Thus, the invention also provides a replicon in which at least part of the nef gene is deleted to create space for the insertion.

To further attenuate a replicon according to the invention, further elements of the wild-type virus may be functionally deleted. Thus, the invention further provides a replicon according to the present invention in which at least one NF-kB element has been deleted. It is preferred that a motif to be activated is a tetO motif, preferably present in an LTR. Thus, the invention also provides a replicon, which comprises at least one tetO motif in at least one functional LTR. It is preferred to have more than one element before an essential gene. Thus, the invention also provides a replicon which comprises at least 2, 4, 6, or 8 such elements in at least one functional LTR. The LTR is preferably modified to avoid reversion to wild type virus.

The invention further provides methods using the replicons to produce dependent viruses, meaning viruses needing an inducing agent in order to replicate. Thus, the invention provides a method for producing a virus dependent on an inducing agent for replication, comprising providing a permissive cell with a replicon according to the invention, culturing the cell in the presence of the inducing agent, and harvesting the dependent virus from the culture. Again, such methods are preferably applied to HIV. Thus, the invention provides a method in which the dependent virus is a human immunodeficiency virus, preferably an attenuated virus.

The preferred inducing agent is again doxycycline. Thus, yet another preferred embodiment is a method in which the inducing agent is doxycycline or a functional analog thereof. Also, part of the present invention includes producing viruses which are produced by the methods or which can be produced by the methods of the present invention. Thus, the invention also provides a virus dependent on an inducing agent for replication obtainable by a method according to the invention, preferably a human immunodeficiency virus that is preferably attenuated.

The viruses will find an important application in vaccination. Therefore, the invention also provides a vaccine comprising a replicon according to the present invention and/or a virus according to the present invention, an amount of the inducing agent, and optionally a suitable adjuvant well known to those of ordinary skill in the art.

The vaccine may comprise a single dosage unit, but may also comprise the inducing agent separately, or it may be made on the spot from a replicon and/or virus that is reconstituted with a liquid excipient such as saline, optionally together with an adjuvant and/or an inducing agent. Viral vaccines are well known in the field. General rules of thumb applicable to known vaccines also apply to the vaccines of the present invention. Doses will be found through the normal dose finding studies performed during (pre)clinical trials, for example, by simple titration of the amount of doxycycline as the inducing agent. The vaccine may be sufficient on its own, but may also be used in addition to other vaccines. The inducing agent may be needed over a longer period of time and can then be provided separately. Again, the preferred vaccine is one for prophylaxis of infection with a human immunodeficiency virus.

The invention also provides the use of the vaccine in that it provides a method for the prohylaxis of AIDS, comprising administering a vaccine according to the invention to a subject, and allowing for viral replication for a limited time by providing the inducing agent. Booster vaccinations are possible by simple readdition of the the inducing agent at later times.

The invention also provides a method for the controlled replication of a virus or a viral replicon comprising providing a permissive cell with a replicon or a virus according to the present invention, culturing the cell in the presence of the inducing agent, and manipulating the amount of inducing agent present.

### BRIEF DESCRIPTION OF THE DRAWINGS

FIG. 1. Design of a Tetracycline-dependent HIV. Panel A shows the HIV-1 genome and multiple modifications that were introduced to construct HIV-rtTA. Details of the mutations are provided in the text (See also panels B and C for the LTR modifications). The TAR-Tat transcriptional axis was inactivated and replaced by the Tetracycline-inducible tetO-rtTA system. Inactivation of TAR and Tat is marked by crosses through the motifs. The genome maps are not drawn to scale, but the genome size of HIV-rtTA is larger than that of HIV-1. The RNA genome of HIV-1 LAI is 9229-nt and HIV-rtTA is 9767-nt (S.S variants) or 9875-nt (K.K variants). Panel B provides some details of the tetO insertions in the LTR promoter. The U3 region of the wild-type LTR (left) encodes 2NF-kB sites (squares) and 3 Sp1 sites (circles). The modified LTR (right) contains either 6 or 8 tetO operators (triangles) upstream of the Sp1 sites. The 6 tetO variant only has the Sp1 sites in mutant S, whereas both NF-kB sites are present upstream of the 8 tetO operators in mutant K. The arrow marks the transcription start site at the U3-R border, which is also the start site of the TAR hairpin. Panel C shows the TAR hairpin structure and the inactivating mutations that were introduced in the bulge (triple-nucleotide substitution) and in the loop (two point mutations). These mutations should disrupt binding of the viral Tat protein and the cellular cyclin T co-factor, respectively (Dingwall et al., 1989; Wei et al., 1998).

FIG. 2. Doxycycline-controlled replication of the HIV-rtTA viruses. The SupT1 T cell line was electroporated with 10 μg of the indicated molecular clones, and cells were cultured in culture medium without dox or with an increasing concentration of dox (0 to 1000 ng/ml range). Virus production was measured by CA-p24 elisa on culture supernatant samples.

FIG. 3. Doxycycline-dependent replication of HIV-rtTA viruses in primary cells. PBMCs were electroporated with the four individual HIV-rtTA constructs (20 μg), and the cultures were maintained without dox or with (1000 ng/ml) dox. Fresh uninfected cells were added immediately after transfection and at day 6 post infection. Virus production was measured by CA-p24 elisa on culture supernatant samples.

FIG. 4. Replication of HIV-rtTA can be turned on and turned off. These experiments were performed with the SWS virus, but similar results have been obtained with the other HIV-rtTA variants. The SWS virus was used (2200 ng CA-p24) to infect 6×10⁶ SupT1 cells at day 0. Panel A shows the replication potential with 0, 100 and 1000 ng/ml dox.. In panel B, the effect of delayed addition of dox (1000 ng/ml) was analyzed at day 3 after infection. In panel C, the infected cells were grown in 1000 ng/ml dox for 3 days, at which point the cells were washed and incubated in the absence or presence of dox. In panel D, infected cells were maintained in the presence of dox and the effect of the Protease-inhibitor Saquinavir (200 nM) and the RT-inhibitor AZT (1 μg) was tested.

FIG. 5. Overview of the original and evolved tetO configuration. The natural situation in Escherichia coli, the situation in most dox-controlled gene expression cassettes with multiple tetO motifs (8× in HIV-rtTA), and the configurations that were selected by spontaneous virus evolution are shown. The latter form either has 2 tetO motifs or 2 tetO motifs with an altered spacing (See FIG. 6 for further details).

FIG. 6. Sequence of the modified tetO configuration was selected by spontaneous virus evolution. The wild-type (wt) sequence of 3 tetO motifs is shown on top. Most viruses evolve from having 8 tetO motifs to having 2 tetO motifs (FIG. 5). The virus cultures are listed that showed a deletion in the tetO-region. A 14-bp deletion was seen in 6 cultures, and a 15-bp deletion was observed in the C6 culture. As indicated, all deletions resulted in removal of the tetO-spacer element.

FIG. 7. The titer of HIV-1 variants with a different tetO configuration. The tissue culture infectious dose (TCID50) on the SupT1 T cell line was determined for several viruses: wild-type HIV-1 (LAI isolate), a nef-deleted LAI variant (LAI nef), HIV-rtTA with 2 tetO motifs and altered spacing (2Δ14), the HXB2 isolate (vpr/vpu/nef-minus), and the original HIV-rtTA construct with 8 tetO motifs. The change from the 8 tetO configuration to the 2Δ14 tetO configuration improved the virus titer approximately 100-fold. On the other hand, wt LAI is 100/1000-fold better than 2Δ14. It is therefore likely that further improvement of 2Δ14 will take place. In fact, HIV-rtTA variants that replicate comparable to wild-type LAI were selected, and the observed rtTA changes were likely responsible for the comparable replication.

FIG. 8. The improved tetO configuration allows for long-term gene expression. The T cell line SupT1 was infected with HIV-rtTA with different tetO configurations (8, 2 and 2Δ14). Gene expression was induced with dox at different times post-infection (day 2, week 2, and week 5). The 8 tetO virus demonstrates complete silencing within 2 weeks. The 2 tetO virus, and in particular the 2Δ14 virus, exhibited sustained activity. The 2Δ14 virus has been shown to be inducible for up to 12 weeks postinfection (not shown).

### DETAILED DESCRIPTION

As stated previously herein, the replication of a viral replicon was put under control of a repressor and/or activator system. In the examples, this was done by incorporation of the Tet-system into the HIV-1 genome.

Several eukaryotic systems for inducible gene expression have been reported, but the Tet-induced regulatory system has some unique properties for incorporation into HIV-1 (Gossen et al., 1993; Gossen & Bujard, 1992; Baron et al., 1999). This Tet-system has found wide application, and strict and graded regulation of gene expression has been reported in many experimental set-ups, such as in the breeding of transgenic animals and gene therapy approaches. Another advantage of these well-characterized regulatory elements from an evolutionary distant organism such as E. coli is that a truly monospecific regulatory circuit in higher eukaryotic cells may be established, thereby limiting the danger of unwanted side effects. This system is based on two elements from the E. coli tet operon, the tetracycline-inducible repressor protein (TetR) that has been converted into a eukaryotic transcriptional activator (tTA or rtTA) and the tetO operator DNA sequence. A novel strategy to impose regulation on HIV-1 gene expression and replication with the Tet-system, such that an exogenous agent (dox), that can be used to reversibly turn on and off viral replication is disclosed in the present invention.

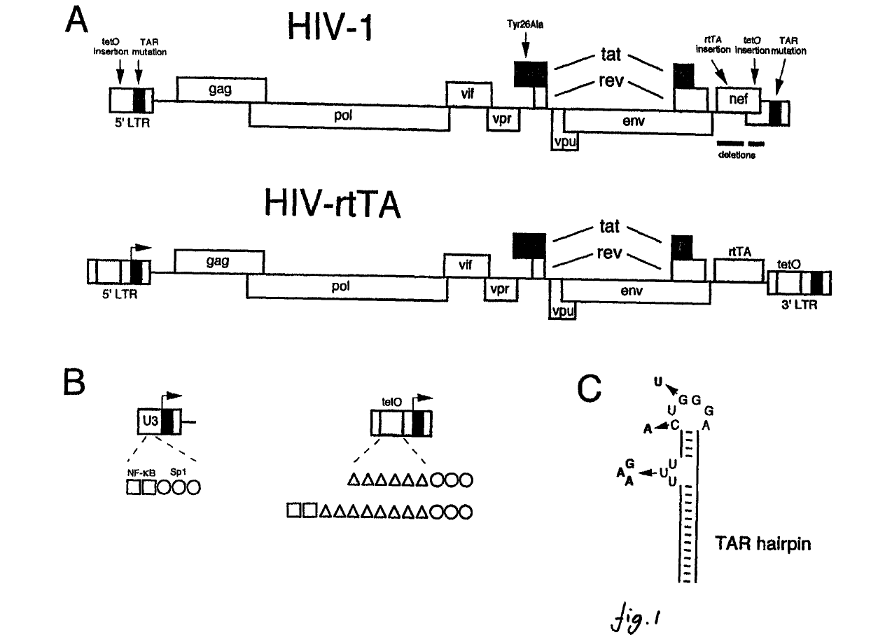

Construction of HIV-rtTA viruses. The full-length, infectious HIV-1 molecular clone pLAI was used to construct an HIV-rtTA virus genome in which the TAR-Tat axis (in FIG. 1A) was replaced by the TetO-rtTA elements. In general, a conservative approach was taken with regard to the types of mutations that were introduced in the HIV-1 genome in order to minimize the chance unknown replicative signals would be inactivated.

TAR and Tat inactivation. First, the TAR element was inactivated by mutating nucleotides in the single-stranded bulge and loop domains (FIG. 1C). A combination of mutations in the bulge and loop domains was chosen because the combination produces a fully inactive TAR motif, while point mutations in one of these single-stranded TAR domains has a dramatic effect on TAR-function in Tat-mediated LTR transcription and virus replication (Berkhout & Jeang, 1991; Berkhout & Jeang, 1989; Berkhout & Klaver, 1993). More gross sequence changes or deletions were not introduced in TAR because the TAR sequence is essential for virus replication as a repeat-R region during strand transfer of reverse transcription (Berkhout et al., 1995). Although it has been demonstrated previously that the TAR element of the 5′LTR is inherited in both LTRs of the viral progeny, the inactive TAR motif was inserted in both LTRs to minimize the chance of a reversion to the wild-type virus by a combination event (Klaver & Berkhout, 1994).

Inactivation of the Tat protein was accomplished by introduction of the Tyr26Ala point mutation. This single amino acid change results in a complete loss of Tat transcriptional activity and viral replication capacity (Verhoef et al., 1997). The corresponding codon change (UAU to GCC) was designed to restrict the likelihood of a simple reversion to the wild-type amino acid, which would require at least two substitutions (Verhoef & Berkhout, 1999). It has been suggested that Tat may play additional roles in the replication cycle besides the transcriptional function (Huanget al., 1994; Harrich et al., 1997; Ulich et al., 1999). Thus, Tat may facilitate HIV-rtTA replication even in the absence of an intact TAR element. Therefore, viruses were also made with the wild-type tat gene and these constructs will be referred to as Y (tyrosine mutant) and W (wild-type).

rtTA and tetO insertion. Two deletions were introduced in the nef gene to create space for the insertion of the non-viral elements (FIG. 1A). A 250-nt upstream fragment and a 200 nt fragment overlapping the U3 region of the 3′LTR were removed. This U3-deletion will be inherited by the viral progeny in both LTRs. The exact borders of the U3 and Nef deletions were carefully chosen such that important cis-acting sequences for virus replication were not removed. In particular, approximately 80-nt around the 5′ end of the 3′LTR was maintained (FIG. 1A). This region encodes multiple sequence elements that are critical for reverse transcription (Ilyinskii & Desrosiers, 1998) and integration (Brown, 1997). In fact, the deletions were an attempt to mimic spontaneous deletions that have been observed in the nef/U3 region of several HIV and SIV variants in a variety of replication studies, including in vivo experiments (Kirchhoff et al., 1994; Fisher & Goff 1998; Ilyinskii et al., 1994; Kirchholl et al., 1995). As preparation for the insertion of the exogenous rtTA gene into the position of the nef gene, a short synthetic sequence that provides a translational start codon in an optimal sequence context (CCAUGU, (Kozak, 1989) and convenient restriction enzyme recognition sites were inserted. The rtTA gene was inserted as a XcmI-XbaI fragment in this polylinker segment in frame with the optimized start codon. The splice acceptor that is located just upstream of the nef gene was maintained such that rtTA translation should occur from the subgenomic mRNA that was originally meant for expression of the Nef protein.

To identify the optimal configuration of an LTR promoter with the rtTA-responsive tetO elements, transient transfection studies were performed with a variety of LTR-luciferace constructs (Verhoef et al., manuscript in preparation.) The number of tetO motifs (2, 4, 6, or 8) that were inserted upstream of the three Sp1 binding sites of the HIV-1 LTR promoter was varied. Constructs with and without the two upstream NF-kB elements were also tested. The two promoters that provided the most robust dox-induced transcription were selected for insertion into the HIV-1 genome and these LTRs are schematically depicted in FIG. 1B. The two promoters will be referred to as K (NF-kB+8 tetO+Sp1) and S (6 tetO+Sp1). Although insertion into the U3 region of the 3′LTR will be sufficient to produce a mutant progeny, the tetO motifs were also introduced in the 5′LTR to generate molecular clones such that the initial round of gene expression in transfected cells will also be regulated in a dox-dependent manner. Thus, both LTRs were modified in both the wild-type and mutant Tat background, resulting in four HIV-rtTA constructs: KWK, KYK, SWS, and SYS. All HIV-rtTA molecular clones have the TAR inactivation and rtTA insertion in common, but the HIV-rtTA molecular clones differ in the status of the tat gene and the type of tetO insert. Of these virus variants, KWK is most wild-type-like because it maintained the NF-kB sites and a wild-type Tat protein, while the variant SYS is the most minimal HIV-rtTA version.

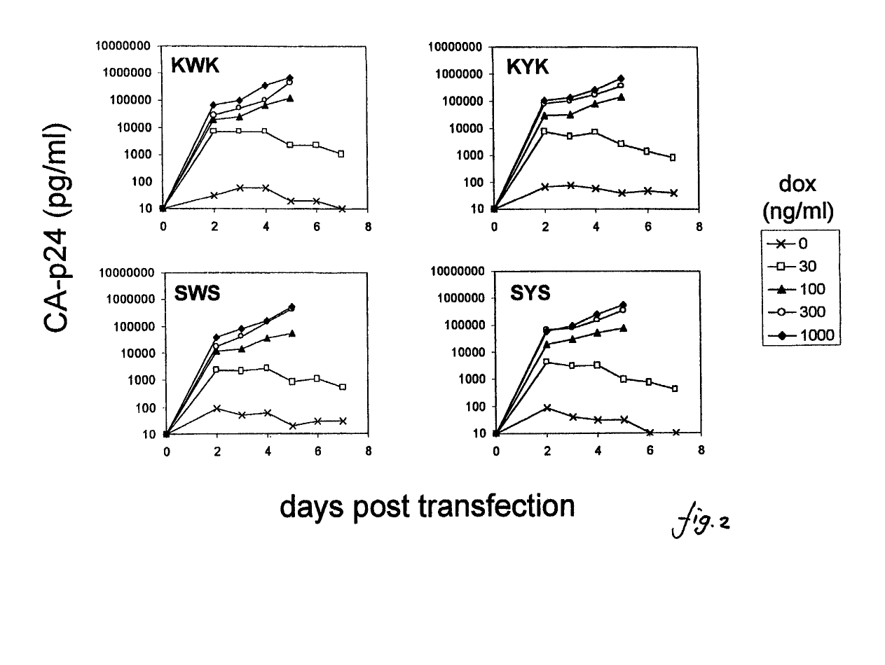

HIV-rtTA replicates in a doxycycline-dependent manner. The four pLAI plasmids were individually transfected into the SupT1 T cell line to test for their replication capacity. Cultures were maintained at varying dox levels, and virus replication was monitored by measuring the amount of CA-p24 produced in the culture medium (FIG. 2). In the presence of optimal dox levels (1000 ng/ml), profound replication of all four HIV-rtTA viruses was measured. No virus replication was observed in the absence of dox, indicating that replication is strictly dependent on the inserted Tet-system. The Tet-system is ideally suited to modulate the level of transcriptional activation in a step-wise manner by reducing the amount of dox (Baron et al., 1997). Indeed, replication of the HIV-rtTA viruses may also be modulated at sub-optimal concentrations of the inducing dox reagent (FIG. 2). A progressive reduction in replication rates of all four rtTA-viruses was observed at 300 and 100 ng/ml dox, and virus replication was nearly abolished at 30 ng/ml. These combined results demonstrate that the HIV-rtTA viruses replicate in a strictly dox-dependent manner and that the rate of replication can be fine-tuned by simple variation of the dox-concentration.

The transfected SupT1 cells were killed within 1 week by the formation of massive virus-induced syncytia, and CA-p24 production levels reached values that are similar to what is observed in regular infections with the wild-type LAI virus. Nevertheless, the HIV-rtTA variants had a significantly reduced fitness because they showed delayed replication in transfections with less DNA (results not shown). Although the four viruses appeared to have a similar replication capacity, this can be measured more appropriately in subsequent infection studies. Indeed, all four HIV-rtTA viruses were passaged as cell-free inoculum onto fresh, uninfected T cells where a spreading infection was sustained for at least 5 weeks (5 passages). From these infection experiments, the following ranking order of replication was apparent: KWK>KYK, SWS>SYS.

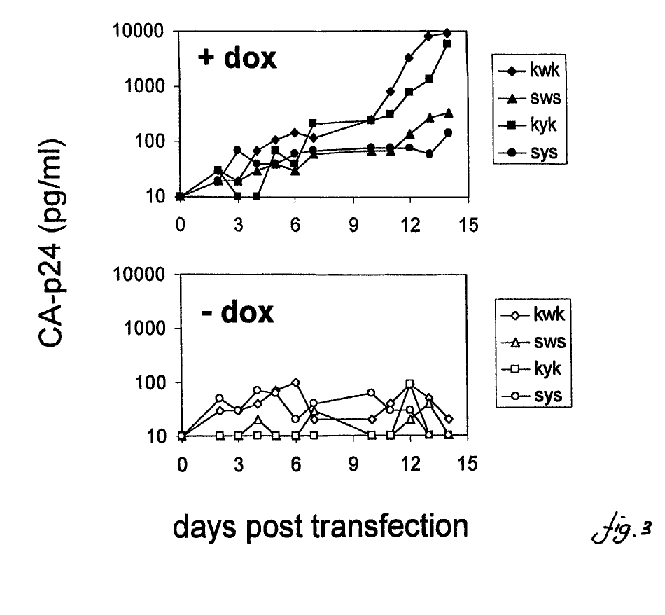

HIV-rtTA vaccine viruses should be able to replicate in primary cells. The LAI molecular clone used in these studies represents a primary isolate that is able to efficiently infect primary cells (Wain-Hobson et al., 1991; Peden et al., 1991), but a complication of our design is that the nef gene was removed. The removal of the nef gene contributed to virus replication in primary cell types (de Ronde et al., 1992). Pooled pheripheral blood mononuclear cells (PBMC) were transfected by means of electroporation with 20 μg of the molecular clones and CA-p24 production in the culture supernatant was measured for up to two weeks (FIG. 3). All four HIV-rtTA variants replicated in the presence of 1000 ng/ml dox, whereas no replication was detectable without dox. The ranking order of replication in PBMCs (KWK>KYK>SWS>SYS) was very similar to that observed in the SupT1 cells.

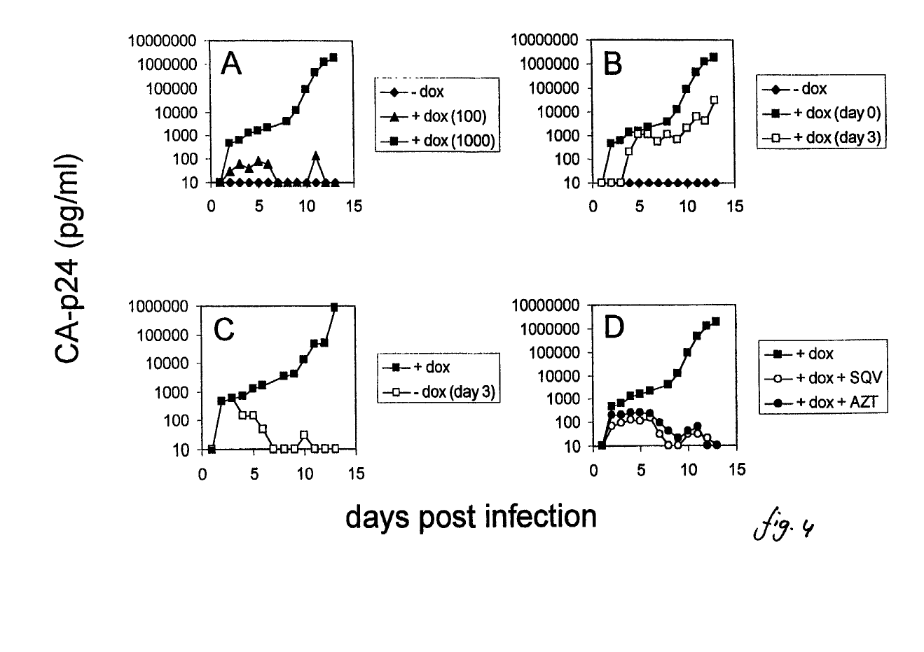

Turning virus replication on and off in a reversible manner. Subsequent tests were performed with the SWS virus in SupT1 infections (FIG. 4). First, the dox-response experiment was repeated. In this more sensitive infection experiment, that the sub-optimal amount of 100 ng/ml dox allowed only a low level of replication that was not sufficient to support a spreading infection (FIG. 4A). Next, virus replication kinetics were analyzed when dox was added 3 days after infection of the cells (FIG. 4B). This resulted in a delay of virus production of approximately 3 days. In the absence of dox, the HIV-rtTA virus can still infect cells, reverse transcribe its RNA genome, and integrate the DNA into the host genome. In other words, the provirus form can be established, where the latently infected cell will remain in the culture and may be activated by dox after three days. An additional feature of the Tet-system is that it provides reversible regulation which was tested in the replication assay (FIG. 4C). In the replication assay, SupT1 cells were infected with the SWS virus and cultured in the presence of dox. At day 3, the cells were washed to remove extracellular dox and resuspended in medium either with or without dox. Indeed, replication can be stopped abruptly by the removal of dox. These combined results confirm that replication of the HIV-rtTA virus is absolutely dependent on dox and that the level of virus replication can be strictly controlled in a graded and reversible manner.

Safety issues. Several assays were performed to analyze different safety aspects of the HIV-rtTA variants. First, leaky virus replication was screened in the absence of dox. For instance, the cell cultures that were transfected with the four different HIV-rtTA constructs (FIG. 2) were maintained without dox for a prolonged period of time, but no virus production was measured in these four cultures up to day 52, at which point the experiment was stopped. Similarly, no replicating virus was observed in primary cells without dox (FIG. 3). In addition, SupT1 cultures in which virus spread was ongoing in the presence of dox were ‘turned off’ by the removal of dox (See e.g., FIG. 4C for the SWS virus) without any sign of virus production. It will be appreciated that these experiments may be viewed as the first safety tests for these vaccine strains.

As an additional safety test, the sensitivity of the HIV-rtTA virus to antiretroviral drugs that are in current clinical use was analyzed. Because the basic set of viral genes in HIV-rtTA was not altered, including the genes encoding Protease (Pro) and Reverse Transcriptase (RT), these viruses are expected to remain fully sensitive to well-known drugs that target these essential enzymes. As shown in FIG. 4D, replication of the dox-dependent SWS virus can be inhibited efficiently either by 3′-azido, 3′-deoxythymidine (AZT, a nucleoside RT-inhibitor) or Saquinavir (SQV, a Pro-inhibitor).

Long-term maintenance of the introduced tetO-rtTA elements. Several important observations have been made with respect to the safety of the HIV-rtTA designer virus. A key issue is whether the HIV-rtTA virus is genetically stable in terms of maintaining the introduced tetO-rtTA system. To see if the HIV-rtTA virus was genetically stable, the virus was passaged for a prolonged time (up to 20 weeks in tissue culture) and monitored in multiple independent cultures for the status of the inactivated Tat-TAR elements and the introduced rtTA-tetO elements. Sequence analysis revealed no repair of either the Tat protein or the TAR RNA element in any of the cultures. Furthermore, the new rtTA-tetO elements were preserved in all samples. These results, combined with the strict dox-dependency of the cultured viruses, demonstrate that the HIV-rtTA virus retained the introduced transcriptional regulatory system.

Extremely low uninduced HIV-1 expression due to the establishment of an autoregulatory loop. The virus replication experiments indicate that gene expression of HIV-rtTA is strictly dependent on dox, which may come as a surprise because most systems for inducible gene expression, including the original rtTA-system, are known to yield a significant level of ‘leaky’ expression in the uninduced state. The superior performance of HIV-rtTA may be due, at least in part, to the use of the modified rtTA variant with reduced ‘leaky activity’. However, it is proposed that the HIV-rtTA system is different from regular dox-controlled gene expression systems in that an autoregulatory loop has been established that reduces the level of leaky gene expression. Specifically, the rtTA expression was placed under the control of an rtTA-regulated LTR promoter, a situation that mimics the natural autoregulatory loop of the TAR-Tat axis. This means that both the activity and the synthesis of rtTA are dox-dependent. Thus, only minute amounts of rtTA protein will be present in the absence of dox, resulting in an extremely low basal level of gene expression and consequently a more profound dox-induction. In many other dox-controlled gene expression systems, the tTA or rtTA protein is produced in a constitutive manner from a second locus, such as the CMV-rtTA plasmid, which causes a significant level of gene activation in the off-state.

An experiment was designed to critically test whether an autoregulatory loop is established in HIV-rtTA. The regular system was mimicked by co-transfection of the HIV-rtTA with CMV-rtTA. The latter plasmid will produce a constitutive level of rtTA protein (even in the absence of dox), which is expected to enhance the level of virus production in the uninduced state. This is indeed what was observed (Table 1). The uninduced level of virus production was increased 5- to 10-fold with CMV-rtTA. The results in Table 1 also indicate that additional synthesis of rtTA protein from the co-transfected CMV-rtTA plasmid does not increase the level of virus production in the presence of dox, indicating that all HIV-rtTA constructs are able to produce an optimal amount of rtTA trans-activator. Due to the increased basal expression levels in co-transfections with CMV-rtTA, only 8- to 16-fold dox-induction levels were measured. An even more profound dox-effect was measured in the T cell line SupT1 (Table 2) which ranged from 390- to 3900-fold induction for the different HIV-rtTA constructs. The combined effects of the autoregulatory loop established in HIV-rtTA and the T cell-specific augmentation of the dox-response resulted in rather dramatic induction levels. In SupT1 cells, an extremely low basal level of virus production was measured and estimated to be approximately 0.03% to 0.2% of the dox-induced state. These results are fully consistent with the inability to measure any virus replication without dox.

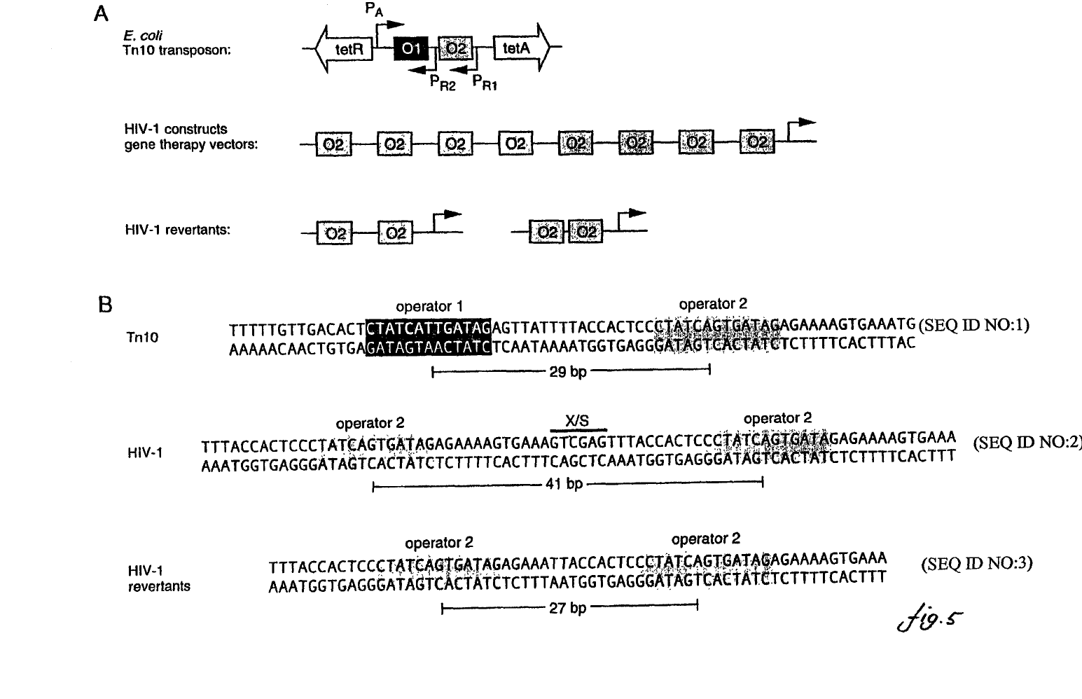

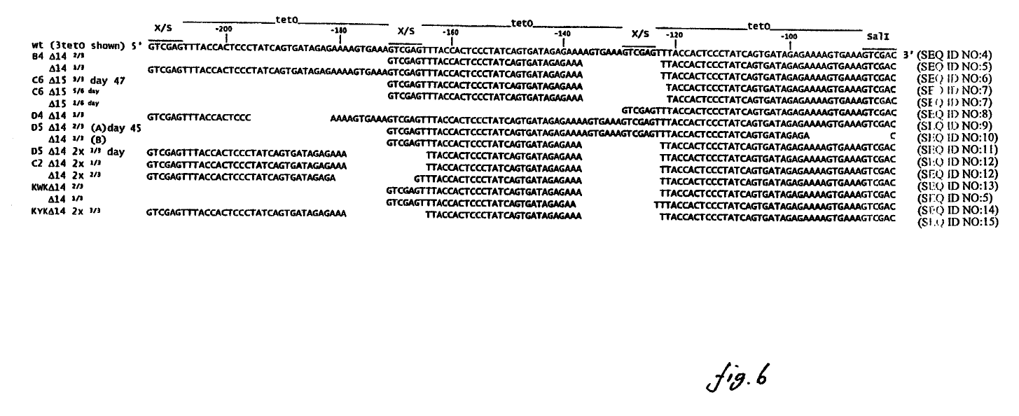

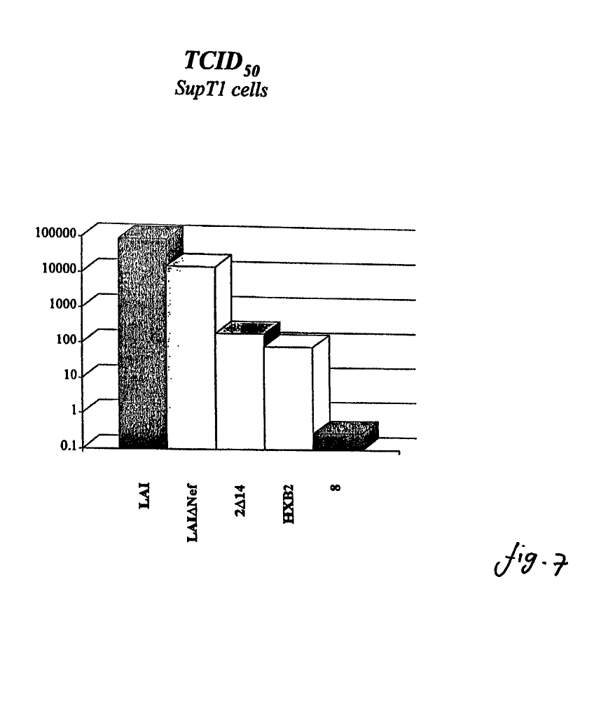

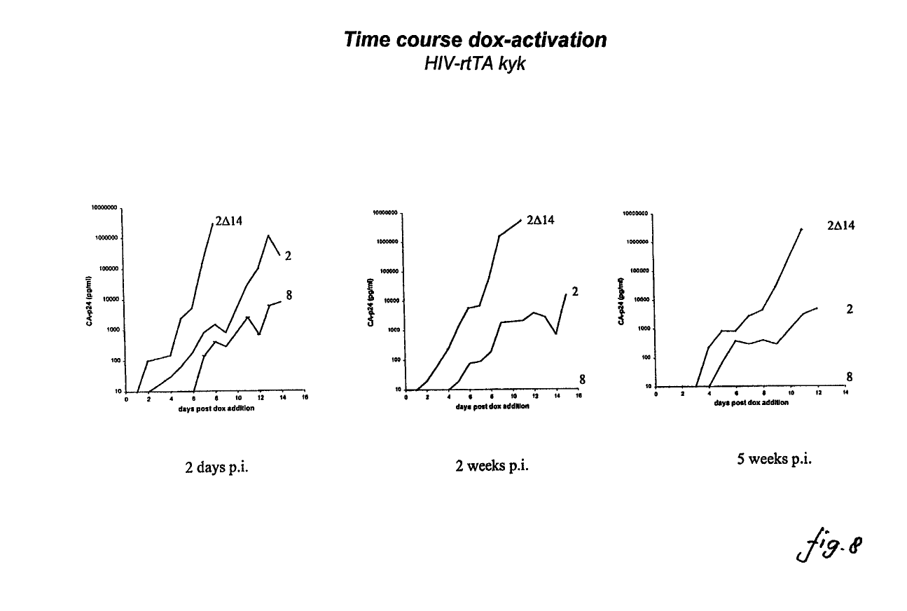

Evolutionary improvement of the Tet-system and improved tetO configuration. The introduced rtTA-tetO elements can be improved/modified by spontaneous virus evolution. Most strikingly, changes in the number and spacing of the individual tetO motifs in many HIV-rtTA evolution experiments were observed (FIG. 5 and FIG. 6). Also, it has been subsequently shown that these modified promoters are responsible for the significant improvement of virus replication that were witnessed over time (FIG. 7). The LTR configuration with 2 tetO motifs and altered spacing was most robust as a dox-regulated promoter when tested in the context of an integrated provirus. This situation reflects not only a natural HIV-1 infection, but also the actual situation of a stably transduced transgene. These findings indicate that a novel tetO configuration has been identified that is optimized for regulated gene expression from a chromosomal position, which occurs in many gene therapy protocols, transgenic mice etc. Furthermore, whereas the original LTR promoter with 8 tetO elements was rapidly silenced within 2 weeks, sustained activity for the LTR promoter with the optimized tetO elements upon dox-induction were measured (FIG. 8).

Improved and modified rtTA. Similarly, improved versions of the rtTA protein were selected. In long-term cultures of HIV-rtTA, changes in well-conserved amino acid residues in important protein domains were observed, including the dox-binding site and the DNA binding site. Many properties of this E. coli protein are the target for evolutionary improvement, including protein stability in the eukaryotic environment, creation of a nuclear import signal, etc. The improvement that was documented for the tetO motifs demonstrated the enormous potential of this viral evolution approach to improve these signals and supports the idea that rtTA variants with a modified effector-specificity may be selected. This includes tetracycline-like effector molecules that do not have antibiotic activity.

The opposite HIV-tTA virus. The HIV-tTA virus variant was also constructed, in which the Tat-TAR axis was replaced by the tTA-tetO system. Again, the replication of this virus is fully dependent on the introduced components of the tetO-rtTA system, but the regulation is opposite to that of HIV-rtTA. The tTA protein is in the DNA-binding conformation without dox and efficient virus replication in this situation was detected. This virus can be selectively and specifically inhibited by dox, which induces a conformational switch in the tTA protein that abrogates its DNA-binding activity. This HIV-tTA reagent is a useful extension of this approach for certain applications. For instance, the tTA-system is ideally suited for gene therapy approaches that require constitutive expression of the transgene, while providing the option to silence transgene expression at a later time by dox-administration. The replicating HIV-tTA reagent also provides a way to improve the tTA reagent by spontaneous virus evolution.

Discussion. The Tet-transcriptional system has been incorporated in the HIV-1 genome such that virus replication can be controlled from the outside by the addition of a non-toxic inducer molecule such as doxycycline (dox). Specifically, replicating HIV-1 variants were constructed with inactivating mutations in both arms of the Tat-TAR axis through replacement with the rtTA-tetO elements of the Tet-system. Replication experiments in a T cell line and primary cells demonstrated that dox-dependent HIV-1 variants were successfully designed. Replication of these designer HIV-rtTA viruses was shown to be regulated in a graded and reversible manner. Although ‘leakiness’ has been a problem in some protocols using the rtTA system, no virus replication was observed in the absence of dox. One possible explanation for the lack of virus replication is that expression of the rtTA trans-activator in the HIV-rtTA system is fully dependent on the presence of dox. Thus, an autoregulatory loop may have been established that resembles the natural TAR-Tat axis. This mechanism may restrict leakiness or dox-independent replication, thereby providing a significant additional safety feature.

The HIV-rtTA viruses have some unique properties that make them ideal reagents for a variety of biological experiments. One application for such a virus is in the field of live-attenuated vaccines, and a similar approach may be used to put control over other retroviral pathogens (e.g., HIV-2, HTLV-I), pararetroviruses (e.g., HBV), or DNA viruses (e.g., herpes virus or adenovirus). The HIV-rtTA viruses improve the current generation of live-attenuated HIV-1 variants as potential vaccine strains because the conditional replication adds a unique safety feature. For instance, the SYS variant has the most minimal ‘genotype’: TAR⁻, Tat⁻, delta-U3, delta-NF-kB, delta-nef, but it should also be possible to delete some of the ‘accessory’ genes such as vpr, vpu and/or vif in addition to the other deleted genes. Therefore, HIV-rtTA vaccine viruses should be able to induce a protective immune response after which replication of the virus can be turned off, such that the virus will be stably non-pathogenic. The HIV-rtTA viruses can still be inhibited by antiviral drugs that are in clinical use as was described previously herein for the RT-inhibitor AZT and the Pro-inhibitor Saquinavir. The HIV-rtTA viruses await extensive replication tests to verify their genetic stability, followed by animal tests to screen for their pathogenic potential and their ability to induce a protective immune response.

Because the TAR RNA and tat gene may have become non-essential parts of the HIV-rtTA genome, these elements may now be ‘free’ to evolve. If these elements have indeed no other function in the viral replication cycle, one would predict that they would eventually be lost by the accumulation of mutations and/or deletions. This further reduces the likelihood of a wild-type-like reversion, thereby making the vaccine strain more safe. However, the situation may be more complex as additional roles have been proposed for both the TAR RNA and tat gene motifs. For instance, the TAR motif is part of the R (repeat) region and is critical in strand transfer during reverse transcription. Additionally, TAR has been reported to contribute to RNA packaging in virion particles (reviewed in Berkhout, 1999). The Tat protein has also been implicated in non-transcriptional roles, e.g., during mRNA translation and the process of reverse transcription (SenGupta et al., 1990; Huang, Joshi, Willey, Orenstein, and Jeang, 1994; Harrich, Ulich, Garcia-Martinez, and Gaynor, 1997; Cullen, 1986). Prolonged culture experiments and the analysis of revertant viruses will provide more insight into some of these possibilities.

The HIV-1 TAR-Tat axis was successfully replaced by the tetO-rtTA system, wherein the latter elements have become essential viral functions. This adds an important safety feature because it precludes the spontaneous loss of the new viral elements by deletion, an event that occurs frequently with exogenous sequences that are inserted in a retroviral genome. Thus, this feature enhances the genetic stability of vaccine strains based on HIV-rtTA. On the other hand, the current HIV-rtTA variants do not yet replicate optimally which is particularly true for the most minimal SYS variant that lacks a functional Tat gene and NF-kB sites. However, continued replication has led to improvement of this new HIV-1 transcriptional axis by selection of spontaneous up-mutants. The beauty of working with HIV and other RNA viruses is that even if a poorly replicating virus is identified, the error-prone nature of the reverse transcriptase (RT) enzyme allows for the generation of faster-replicating variants by a method termed forced evolution (Klaver & Berkhout, 1994; Berkhout & Das, 1999). This evolutionary refinement of the initial designer HIV-rtTA variants provides a powerful method to select for fast-replicating, dox-dependent HIV-1 variants. Using this evolutionary approach, modified forms of the rtTA protein and the tetO sites were selected for that are better suited for their new role in virus replication. Thus, the invention also provides a method for modifying an inducible replicon which includes generating a viral replicon comprising a nucleic acid encoding all viral sequences that are essential for replication, wherein the nucleic acid is under direct or indirect control of at least one inducible repressor and/or activator. The method also includes providing cells permissive for replication of the replicon using the replicon, culturing the cells under conditions that allow for the replication of the replicon, and obtaining replicated replicons from the culture. The replicon may be derived from an infectious human immunodeficiency virus clone. As described herein, this method is well suited for obtaining a modified repressor, activator and/or promoter. Thus, the invention also provides a nucleic acid encoding a repressor and/or activator obtainable by the method. The invention also provides a promoter obtainable by the method.

In yet another embodiment, the present invention discloses a cell comprising a replicon of the invention. The replicon may be modified by a method of the invention described in the preceding paragraph. A cell may also be provided with a modified repressor, activator and/or promoter. Therefore, the invention also discloses a cell comprising a nucleic acid encoding a repressor and/or activator obtainable by the method. The invention also discloses a cell comprising a promoter obtainable by the method.

It is expected that the enormous evolutionary capacity of HIV-1 can be used to select for rtTA elements with altered substrate-specificity by gradually changing dox or other dox-like derivatives in the culture medium. Thus, the virus will help us to find better tetO-rtTA reagents that can subsequently be useful in biological settings that require specific regulation of gene expression (e.g., transgenic mice, gene therapy). We plan to rigorously test the possibility to perform genetics with the ‘prokaryotic’ Tet-system in this eukaryotic (viral) background.

Although the novel rtTA-tetO reagents can be used to improve any gene expression system that uses this dox-regulated mechanism, it has been directed herein to the implications for retroviral packaging cell lines and retroviral (gene therapy) vectors. Packaging cell lines based on the HIV-1 lentivirus are notoriously difficult to establish because of the toxicity of some viral proteins, where an inducible system is required. This system may be improved at several levels. First, a 1-plasmid construct has been made that expresses both the rtTA protein and the HIV-1 proteins. Second, because of the autoregulatory loop for rtTA synthesis, this system provides an extremely low level of basal activity (‘leakiness’). Third, the LTR promoter with the novel tetO configuration is more powerful to drive high-level expression. Fourth, this modified LTR is less sensitive to silencing, which is due to chromatin remodelling and/or methylation. The same benefits apply to gene therapy vectors, where improved regulation of transgene expression is critical (either lower basal expression, more robust dox-induced expression, or the absence of transgene-silencing over time). In addition, the tTA-version may be particularly important in long-term transgene expression strategies. Finally, the ability to select for virus variants with rtTA proteins that exhibit either a modified dox-response (e.g., at a lower dox-concentration) or novel effector-specificity may help in the design of additional regulatory systems that allow the independent regulation of multiple transgenes with different effector molecules.

### References

Almond N, Kent K, Cranage M, Rud E, Clarke B, Stott E J. 1995. Protection by attenuated simian immunodeficiency virus in macaques against challenge with virus-infected cells. Lancet 345:1342-4.

Baba T W, Liska V, Khimani A H, Ray N B, Dailey P J, Penninck D, Bronson R, Greene M F, McClure H M, Martin L N, et al. 1999. Live attenuated, multiply deleted simian immunodeficiency virus causes AIDS in infant and adult macaques. Nature Medicine 5:194-203.

Baron U, Gossen M, Bujard H. 1997. Tetracycline-controlled transcription in eukaryotes: novel transactivators with graded transactivation potential. Nucleic Acids Res. 25:2723-9.

Baron U, Schnappinger D, Helbl V, Gossen M, Hillen W, Bujard H. 1999. Generation of conditional mutants in higher eukaryotes by switching between the expression of two genes. Proc. Natl. Acad. Sci. USA 96:1013-8.

Berkhout B. 1999. Multiple biological roles associated with the repeat (R) region of the HIV-1 RNA genome. Adv. Pharmacol. in press:

Berkhout B, Das A T. 1999. Functional analysis of RNA signals in the HIV-1 genome by forced evolution. In:

Barciszewski J, Clark B F C, eds. RNA biochemistry and biotechnology. Dordrecht, the Netherlands.: Kluwer Academic Publishers; pp. in press

Berkhout B, Jeang K T. 1989. Trans activation of human immunodeficiency virus type 1 is sequence specific for both the single-stranded bulge and loop of the trans-acting-responsive hairpin: a quantitative analysis. J. Virol. 63:5501-4.

Berkhout B, Jeang K T. 1991. Detailed mutational analysis of TAR RNA: critical spacing between the bulge and loop recognition domains. Nucleic Acids Res. 19:6169-76.

Berkhout B, Klaver B. 1993. In vivo selection of randomly mutated retroviral genomes. Nucleic Acids Res. 21:5020-4.

Berkhout B, van Wamel J, Klaver B. 1995. Requirements for DNA strand transfer during reverse transcription in mutant HIV-1 virions. J. Mol. Biol. 252:59-69.

Berkhout B, Verhoef K, van Wamel J L B, Back B. 1999. Genetic instability of live-attenuated HIV-1 vaccine strains. J. Virol. 73:1138-45.

Brown P O. 1997. Integration. In: Coffin J M, Hughes S H, Varmus H E, eds. Retroviruses. New York: Cold Spring Harbor Laboratory Press; pp. 161-204.

Cullen B R. 1986. Trans-activation of human immunodeficiency virus occurs via a bimodal mechanism. Cell 46:973-82.

Daniel M D, Kirchhoff F, Czajak S C, Sehgal P K, Desrosiers R C. 1992. Protective effects of a live attenuated SIV vaccine with a deletion in the nef gene. Science 258:1938-41.

de Ronde A, Klaver B, Keulen W, Smit L, Goudsmit J. 1992. Natural HIV-1 NEF accelerates virus replication in primary human lymphocytes. Virol. 178:1-5.

Deacon N J, Tsykin A, Solomon A, Smith K. Ludford-Menting M, Hooker D J, McPhee D A, Greenway A L, Ellett A, Chatfield C. et al. 1995. Genomic structure of an attenuated quasi species of HIV-1 from blood transfusion donor and recipients. Science 270:988-91.

Dingwall C, Emberg I, Gait M J, Green S M, Heaphy S, Karn J, Lowe A D, Singh M, Skinner M A, Valerio R. 1989. Human Immunodeficiency Virus 1 tat protein binds trans-activating-responsive region (TAR) RNA in vitro. Proc. Natl. Acad. Sci. USA 86:6925-9.

Dyer W B, Ogg G S, Demoitie M- A, Jin X, Geczy A F, Rowland-Jones S L, McMichael A J, Nixon D F, Sullivan J S. 1999. Strong human immunodeficiency virus (HIV)-specific cytotoxic T-lymphocyte activity in Sydney blood bank cohort patients infected with nef-defective HIV type 1. J. Virol. 73:436-43.

Fisher J, Goff S P. 1998. Mutational analysis of stem-loops in the RNA packaging signal of the Moloney murine leukemia virus. Virol. 244:133-45.

Gossen M, Bonin A L, Bujard H. 1993. Control of gene activity in higher eukaryotic cells by prokaryotic regulatory elements. Trends Biochem. Sci. 18:471-5.

Gossen M, Bujard H. 1992. Tight control of gene expression in mammalian cells by tetracycline-responsive promoters. Proc. Natl. Acad. Sci. USA 89:5547-51.

Greenough T C, Sullivan J L, Desrosiers R C. 1999. Declining CD4 T-cell counts in a person infected with nef-deleted HIV-1. New Engl. J Med. 340:236-7.

Harrich D, Ulich C, Garcia-Martinez L F, Gaynor R B. 1997. Tat is required for efficient HIV-1 reverse transcription. EMBO J. 16:1224-35.

Huang L M, Joshi A, Willey R, Orenstein J, Jeang K T. 1994. Human immunodeficiency viruses regulated by alternative trans- activators: genetic evidence for a novel non-transcriptional function of Tat in virion infectivity. EMBO J. 13:2886-96.

Ilyinskii P O, Daniel M D, Simon M A, Lackner A A, Desrosiers R C. 1994. The role of the upstream U3 sequences in the pathogenesis of simian immunodeficiency virus-induced AIDS in rhesus monkeys. J. Virol. 68:5933-44.

Ilyinskii P O, Desrosiers R C. 1998. Identification of a sequence element immediately upstream of the polypurine tract that is essential for replication of simian immunodeficiency virus. EMBO J. 17:3766-74.

Johnson R P, Lifson J D, Czajak S C, Stefano cole K, Manson K H, Glickman R L, Yang J Q, Montefiori D C, Montelaro R C, Wyand M S. et al. 1999. Highly attenuated vaccine strains of simian immunodeficiency virus protect against vaginal challenge: inverse relationship of degree of protection with level of attenuation. J. Virol. 73:4952-61.

Kirchhoff F, Greenough T, Brettler D B, Sullivan J L, Desrosiers R C. 1995. Absence of intact nef sequences in along-term survivor with nonprogressive HIV-1 infection. New Engl. J. Med. 332:228-32.

Kirchhoff F, Kestler III H W, Desrosiers R C. 1994. Upstream U3 sequences in simian immunodeficiency virus are selectively deleted in vivo in the absence of an intact nef gene. J. Virol. 68:2031-17.

Klaver B, Berkhout B. 1994. Evolution of a disrupted TAR RNA hairpin structure in the HIV-1 virus. EMBO J. 13:2650-9.

Klaver B, Berkhout B. 1994. Premature strand transfer by the HIV-1 reverse transcriptase during strong-stop DNA synthesis. Nucleic Acids Res. 22:137-44.

Kozak M. 1989. The scanning model for translation: an update. J. Cell Biol. 108:229-41.

Lohman B L, McChesney M B, Miller C J, McGowan E, Joye S M, van Rompay K K, Reay E, Antipa L, Pedersen N C, Marthas M L. 1994. A partially attenuated simian immunodeficiency virus induces host immunity that correlates with resistance to pathogenic virus challenge. J. Virol. 68:7021-9.

Paul W E. 1995. Can the immune response control HIV infection? Cell 82:177-82.

Peden K, Emerman M, Montagnier L. 1991. Changes in growth properties on passage in tissue culture of viruses derived from infectious molecular clones of HIV-1_{LAI}, HIV-1_{MAL}, and HIV-1_{ELI} Virol. 185:661-72.

SenGupta D N, Berkhout B, Gatignol A, Zhou A M, Silverman R H. 1990. Direct evidence for translational regulation by leader RNA and Tat protein of human immunodeficiency virus type 1. Proc. Natl. Acad. Sci. USA 87:7492-6.

Shibata R, Siemon C, Czajak S C, Desrosiers R C, Martin M A. 1997. Live, attenuated simian immunodeficiency virus vaccines elicit potent resistance against a challenge with a human immunodeficiency virus type 1 chimeric virus. J. Virol. 71:8141-8.

Stahl-Hennig C, Dittmer U, Nisslein T, Petry H, Jurkiewicz E, Fuchs D, Wachter H, Matz-Rensing K, Kuhn E M, Kaup F J, et al. 1996. Rapid development of vaccine protection in macaques by live-attenuated simian immunodeficiency virus. J. Gen. Virol. 77:2969-81.

Ulich C, Dunne A, Parry E, Hooker C W, Gaynor R B, Harrich D. 1999. Functional domains of Tat required for efficient human immunodeficiency type 1 reverse transcription. J. Virol. 73:2499-508.

van Rompay K K A, Spinner A, Otsyula M, McChesney M B, Marthas M L. 1995. Attenuated retrovirus vaccines and AIDS. Science 270:1218

Verhoef K, Berkhout B. 1999. A second-site mutation that restores replication of a Tat-defective human immunodeficiency virus. J. Virol. 73:2781-9.

Verhoef K, Koper M, Berkhout B. 1997. Determination of the minimal amount of Tat activity required for human immunodeficiency virus type 1 replication. Virol. 237:228-36.

Wain-Hobson S, Vartanian J- P, Henry M, Chenciner N, Cheynier R, Delassus S, Pedroza Martins L, Sala M, Nugeyre M T, Guetard, et al. 1991. LAV revisited: origins of the early HIV-1 isolates from Institut Pasteur. Science 252:961-5.

Wei P, Garber M E, Fang S- M, Fisher W H, Jones K A. 1998. A novel CDK9-associated C-type cyclin interacts directly with HIV-1 Tat and mediates its high-affinity, loop-specific binding to TAR RNA. Cell 92:451-62.

Wyand M S, Manson K H, Garcia-Moll M, Montefiori D, Desrosiers R C. 1996. Vaccine protection by a triple deletion mutant of simian immuodeficiency virus. J. Virol. 70:3724-33.

TABLE 1
Transient HIV-rtTA production in C33A cells (pg/ml CA-p24)

| | | | | +CMV-rtTA | | |
|---|---|---|---|---|---|---|
| | | | fold | | | fold |
| | −dox | +dox | induction | −dox | +dox | induction |
| KWK | 11,600 | 545,000 | 47 × | 91,500 | 715,000 | 7.8 × |
| SWS | 11,350 | 560,000 | 49 × | 49,500 | 560,000 | 11.3 × |
| KYK | 4,850 | 580,000 | 120 × | 47,500 | 500,000 | 10.5 × |
| SYS | 5,950 | 455,000 | 76 × | 32,500 | 520,000 | 16 × |
| LAI | 635,000 | 520,000 | 0.8 × | — | — | — |

TABLE 2
Transient HIV-rtTA production in SupT1 cells (pg/ml CA-p24)

| | | | fold |
|---|---|---|---|
| | −dox | +dox | induction |
| KWK | 110 | 54,000 | 491 × |
| SWS | 30 | 65,000 | 2167 × |
| KYK | 10 | 39,000 | 3900 × |
| SYS | 20 | 7,800 | 390 × |

1. An inducible viral replicon comprising:
at least one inducible repressor and/or activator; and
at least one viral sequence essential for replication, wherein said at least one viral sequence is under direct or indirect control of said inducible repressor and/or activator.

2. The inducible viral replicon of claim 1, wherein at least one of said at least one viral sequence comprises RNA.

3. The inducible viral replicon of claim 1 or 2, wherein said inducible viral replicon is of a human immunodeficiency virus origin.

4. The inducible viral replicon of claim 3, wherein said inducible viral replicon is of an infectious human immunodeficiency virus clone origin.

5. The inducible viral replicon of any one of claims 1-4, wherein said inducible repressor and/or activator comprises a Tet operon or a functional equivalent thereof.

6. The inducible viral replicon of any one of claims 1-5, wherein said inducible viral replicon encodes an attenuated virus.

7. The inducible viral replicon of any one of claims 1-6 further comprising a suicide gene.

8. The inducible viral replicon of any one of claims 1-7, wherein said inducible viral replicon is induced to replicate by doxycycline or a functional equivalent thereof.

9. The inducible viral replicon of any one of claims 3-8 further comprising an inactivated TAR element.

10. The inducible viral replicon of any one of claims 3-9 further comprising an inactivated Tat element.

11. The inducible viral replicon of any one of claims 3-8, wherein at least one functional part of said inducible repressor and/or activator is inserted into a nef gene.

12. The inducible viral replicon of claim 1 1, wherein at least part of the nef gene is deleted to create space for the insertion.

13. The inducible viral replicon of any one of claims 3-12, wherein at least one NF-kB element has been deleted.

14. The inducible viral replicon of any one of claims 3-13 further comprising at least one tetO motif inserted in at least one functional LTR.

15. The inducible viral replicon of claim 14 further comprising 2, 4, 6, or 8 tetO motifs inserted in said at least one functional LTR.

16. The inducible viral replicon of any one of claims 3-15, wherein at least one LTR is modified to avoid reversion to a wild type virus.

17. A process for producing a virus dependent on an inducing agent for replication, comprising:
providing at least one permissive cell with the inducible viral replicon of any one of claims 1-16;
culturing said at least one permissive cell in the presence of an inducing agent; and
harvesting a dependent virus produced from a culture of said at least one permissive cell.

18. The process of claim 17, wherein said dependent virus comprises a human immunodeficiency virus.

19. The process of claim 17 or 18, wherein said dependent virus is an attenuated virus.

20. The process of any one of claims 17-19, wherein said inducing agent comprises doxycycline or a functional equivalent thereof.

21. A virus dependent on an inducing agent for replication produced by the process of any one of claims 17-20.

22. The virus of claim 21, wherein said virus comprises a human immunodeficiency virus.

23. The virus of claim 21 or 22, wherein said virus is an attenuated virus.

24. A vaccine comprising:
the inducible viral replicon of any one of claims 1-16; and/or
the virus of any one of claims 21-23; and
a suitable adjuvant.

25. The vaccine of claim 24 further comprising an inducing agent.

26. The vaccine of claim 24 or 25, wherein said inducible viral replicon and/or said virus is of a human immunodeficiency virus origin.

27. A method of controlling replication of a virus or an inducible viral replicon comprising:
providing at least one permissive cell with the inducible viral replicon of any one of claims 1-16, or the virus of any one of claims 21, 22 or 23;
culturing said at least one permissive cell in the presence of an inducing agent in a culture; and
manipulating an amount of said inducing agent present in said culture.

28. A method for the prohylaxis of AIDS in a subject, comprising:
administering a vaccine of any one of claims 24-26 to a subject; and
allowing for viral replication for a limited time by providing an inducing agent.

29. A process for producing a replicated replicon using the inducible viral replicon of any one of claims 1-16, comprising:
generating a inducible viral replicon comprising at least one nucleic acid encoding all viral sequences which are essential for replication under direct or indirect control of at least one inducible repressor and/or activator;
providing at least one permissive cell;
culturing said at least one permissive cell with said inducible viral replication under conditions that allow said inducible viral replicon to replicate and form at least one replicated replicon in a culture; and
obtaining said replicated replicon from said culture.

30. A modified nucleic acid encoding a repressor and/or activator produced by the process of claim 29.

31. A modified promoter produced by the process of claim 29.

32. A cell comprising the inducible viral replicon of any one of claims 1-16.

33. A cell comprising the modified nucleic acid of claim 30.

34. A cell comprising the modified promoter of claim 31.


SEQUENCE LISTING (15 sequences)

SEQ ID NO: 1 - DNA, 71 bp, Escherichia coli
tttttgttgacactctatcattgatagagttattttaccactccctatcagtgatagagaaaagtgaaatg

SEQ ID NO: 2 - DNA, 78 bp, Artificial Sequence
  HIV-1 molecular clone pLAI construct
tttaccactccctatcagtgatagagaaaagtgaaagtcgagtttaccactccctatcagtgatagagaaaagtgaaa

SEQ ID NO: 3 - DNA, 64 bp, Artificial Sequence
  HIV-1 Revertant
tttaccactccctatcagtgatagagaaattaccactccctatcagtgatagagaaaagtgaaa

SEQ ID NO: 4 - DNA, 132 bp, HIV-1 molecular clone pLAI
gtcgagtttaccactccctatcagtgatagagaaaagtgaaagtcgagtttaccactccctatcagtgatagagaaaagtgaaagtcgagtttaccactccctatcagtgatagagaaaagtgaaagtcgac

SEQ ID NO: 5 - DNA, 75 bp, Artificial Sequence
  HIV-1 revertant
gtcgagtttaccactccctatcagtgatagagaaattaccactccctatcagtgatagagaaagtgaaagtcgac

SEQ ID NO: 6 - DNA, 118 bp, Artificial Sequence
  HIV-1 revertant
gtcgagtttaccactccctatcagtgatagagaaaagtgaaagtcgagtttaccactccctatcagtgatagagaaattaccactccctatcagtgatagagaaaagtgaaagtcgac

SEQ ID NO: 7 - DNA, 74 bp, Artificial Sequence
  HIV-1 revertant
gtcgagtttaccactccctatcagtgatagagaaataccactccctatcagtgatagagaaagtgaaagtcgac

SEQ ID NO: 8 - DNA, 48 bp, Artificial Sequence
  HIV-1 revertant
gtcgagtttaccactccctatcagtgatagagaaaagtgaaagtcgac

SEQ ID NO: 9 - DNA, 118 bp, Artificial Sequence
  HIV-1 revertant
gtcgagtttaccactcccaaaagtgaaagtcgagtttaccactccctatcagtgatagagaaaagtgaaagtcgagtttaccactccctatcagtgatagagaaaagtgaaagtcgac

SEQ ID NO: 10 - DNA, 76 bp, Artificial Sequence
  HIV-1 Revertant
gtcgagtttaccactccctatcagtgatagagaaaagtgaaagtcgagtttaccactccctatcagtgatagagac

SEQ ID NO: 11 - DNA, 76 bp, Artificial Sequence
  HIV-1 revertant
gtcgagtttaccactccctatcagtgatagagaaattaccactccctatcagtgatagagaaaagtgaaagtcgac

SEQ ID NO: 12 - DNA, 104 bp, Artificial Sequence
  HIV-1 revertant
gtcgagtttaccactccctatcagtgatagagaaattaccactccctatcagtgatagagaaattaccactccctatcagtgatagagaaaagtgaaagtcgac

SEQ ID NO: 13 - DNA, 104 bp, Artificial Sequence
  HIV-1 revertant
gtcgagtttaccactccctatcagtgatagagagtttaccactccctatcagtgatagagaaattaccactccctatcagtgatagagaaaagtgaaagtcgac

SEQ ID NO: 14 - DNA, 75 bp, Artificial Sequence
  HIV-1 revertant
gtcgagtttaccactccctatcagtgatagagaatttaccactccctatcagtgatagagaaagtgaaagtcgac

SEQ ID NO: 15 - DNA, 104 bp, Artificial Sequence; identical to SEQ ID NO: 12
  HIV-1 revertant